(19) United States
(12) Patent Application Publication
(10) Pub. No.: US 2011/0182820 A1
(43) Pub. Date: Jul. 28, 2011

(54) METHODS FOR THE PREDICTION OF SHORT-TERM AND LONG-TERM COGNITIVE DECLINE IN ALZHEIMER'S DISEASE PATIENTS USING CSF BIOMARKERS

(76) Inventors: Jeffrey L. Seeburger, Wayne, PA (US); Daniel J. Holder, Blue Bell, PA (US); A. David Smith, Oxford (GB); Abderrahim Oulhaj, Oxford (GB)

(21) Appl. No.: 13/055,842

(22) PCT Filed: Jul. 16, 2009
(86) PCT No.: PCT/US09/50807

§ 371 Date: Mar. 23, 2011

Related U.S. Application Data
(60) Provisional application No. 61/135,995, filed on Jul. 25, 2008.

(51) Int. Cl. A61K 49/00; G01N 33/53

(52) U.S. Cl. 424/9.2; 435/7.94

(57) ABSTRACT

The present invention provides a method for predicting the short-term and long-term cognitive decline in Alzheimer's disease patients and uses thereof in predicting efficacy of an AD therapeutic. The method uses baseline levels of CSF biomarkers to predict decreases over time in CAMCOG and MMSE scores.

### FIELD OF THE INVENTION

The present invention relates generally to the prognosis of Alzheimer's disease. More specifically, it relates to biomarkers that can be used for the prognosis of cognitive decline in Alzheimer's disease patients.

### BACKGROUND OF THE INVENTION

Alzheimer's disease (AD) is a major neurodegenerative disease of unknown etiology that is characterized by the selective degeneration of basal forebrain cholinergic neurons. The degeneration of these cells leads to a secondary loss of neurons in the limbic system and cortex that control learning and memory. The consequent symptoms of the disease include a progressive loss of memory, the loss of the ability to communicate and the loss of other cognitive functions which occur over a course of approximately eight years. Over the course of this cognitive decline patients often become bedridden and completely unable to care for themselves. Although several symptomatic therapies have been approved to provide some compensation for the cholinergic deficit, for example, Aricept® (donepezil HCl, Eisai Co., Ltd. and Pfizer Inc.), the clinical effects of these are modest and none are able to significantly alter the course of the disease. Improving upon strategies for the treatment of AD has become a focus for the medical and scientific communities due to increases in the average age of the world population, the consequent increase in incidence and prevalence of age-related disorders such as AD, and the severe socioeconomic impact associated with supporting such cognitively impaired patients over the long term.

Requisite to improving the treatment of AD is improving the ability of clinicians to accurately diagnose the disease and to accurately predict the course of the disease. Currently, a diagnosis of possible or probable AD is made based on clinical symptoms. Patients who present with symptoms of memory impairment, but who do not fulfill the clinical criteria for AD, may be given a diagnosis of mild cognitive impairment (MCI). Approximately half of all patients diagnosed with MCI go on to develop AD. A definitive diagnosis of AD can only be made post-mortem and requires a pathological examination of the affected brain tissue. The key pathological hallmarks of the disease are plaques consisting of deposited amyloid beta (Aβ) protein and tangles consisting of degenerated neuronal cells and their cytoskeletal elements (neurofibrillary tangles). Compared to the pathological diagnosis, the pre-mortem clinical diagnosis can achieve an accuracy of approximately 80% to 90%. However, this level of diagnostic accuracy more commonly occurs at well-experienced AD centers and for patients who have been manifesting clinical symptoms for several years (Rasmusson, D. X., et al., Alzheimer Dis. Assoc. Disord., 10(4): 180-188, 1996; Frank, R. A. et al., Proceedings of the Biological Markers Working Group: NIA Initiative on Neuroimaging in Alzheimer's Disease, Neurobiol. Ageing, 24: 521-536, 2003). Following the clinical diagnosis, the course of the disease is typically monitored through cognitive testing and assessment of everyday function. The course is often variable across patients and may be influenced by both organic and environmental elements. There are currently no tests that, in and among themselves, have been validated to identify AD and predict the course of the decline.

The last decade has seen an increase in efforts to identify and validate AD-related biomarkers that might increase the sensitivity and specificity of diagnosis and provide a basis for predicting progression (Regan Research Institute and National Institute of Ageing (NIA) Consensus Report of the Working Group on: ‘Molecular and Biochemical Markers of Alzheimer's Disease,’ Neurobiol. Ageing, 19(2): 109-116, 1998; Frank et al., 2003). Among the techniques that currently hold promise in this regard is the biochemical analysis of cerebrospinal fluid (CSF). The value of CSF analysis is based on the fact that the composition of this fluid may reflect brain biochemistry due to its direct contact with brain tissue.

The CSF proteins that have received the most attention are those thought to reflect key features of the disease pathogenesis, including Aβ deposition and neuronal degeneration. Studies have demonstrated reduced levels of the Aβ42 peptide in the CSF of clinically diagnosed AD patients compared to controls (Andreasen, N., et al., Arch. Neurol., 58: 373-379, 2001; NIA Consensus Report, 1998; Frank et al, 2003, Andreasen, N., et al., Clin. Neurol. Neurosurg. 107: 165-173, 2005). Aβ42 is a cleavage product of the amyloid precursor protein (APP) and is thought to be a major constituent of the senile plaque. One theory of disease progression is that reduced CSF levels in AD patients may be due to increased deposition of the peptide in the brain. In contrast, many studies have shown that the expression of the Aβ40 peptide, another APP cleavage product that is also a plaque component, may be similar in clinically diagnosed AD and control CSF (Frank et al, 2003).

The tau protein is another CSF protein that has been studied for disease etiology. Tau is an axonal protein that, when hyperphosphorylated, assembles into the paired helical filaments that form neurofibrillary tangles. Whereas the presence of tau in the CSF is thought to be a general reflection of axonal (i.e., neuronal) degeneration in the brain, the presence of phosphorylated tau (ptau) may be a more specific indicator of AD-related pathology. CSF levels of both tau and ptau in clinically diagnosed AD patients have been shown in many studies to be elevated compared to that in controls (Andreasen, 2001; and for review, Consensus Report, 1998; Frank et al, 2003 and Andreasen, 2005).

A recent review article describes not only the status of biochemical biomarkers, but also the active area of imaging biomarkers and their use in longitudinal clinical trials (Thal, L. J., et al, Alzheimer Dis. Assoc. Disord., 20(1): 6-15, 2006). It is important to note that generally, the field has focused on diagnostic or prognostic biochemical biomarkers and there are few papers that have successfully identified disease progression markers from fluid samples.

### SUMMARY OF THE INVENTION

In one embodiment the present invention is directed to a method for predicting short-term cognitive decline in an Alzheimer's disease (AD) patient comprising: (a) selecting an Alzheimer's disease patient; (b) conducting an initial prognostic assessment of said patient, where the prognostic assessment comprises a cognitive assessment of the patient and a biomarker analysis of a fluid sample from the patient; (c) comparing the baseline CSF biomarker levels to a statistically significant slope (SSS) obtained from a standard AD patient panel; and, (d) determining the predicted short-term rate of cognitive decline, where the predicted short-term rate of cognitive decline is the predicted decrease in a CAMCOG or MMSE score.

Another embodiment of the invention is directed to a method for evaluating the effectiveness of an AD therapeutic comprising: (a) selecting an Alzheimer's disease patient; (b) conducting an initial prognostic assessment of said patient, where the prognostic assessment comprises a cognitive assessment of the patient and a biomarker analysis of a fluid sample from the patient; (c) comparing the baseline CSF biomarker levels to a statistically significant slope (SSS) obtained from a standard AD patient panel; (d) determining the predicted rate of cognitive decline, where the predicted rate of cognitive decline is the predicted decrease in a CAMCOG or MMSE score; (e) administering an AD therapeutic to AD patient; (f) conducting one or more subsequent prognostic assessments on a periodic basis of each AD patient; (g) determining an actual rate of cognitive decline in the AD patient; and (h) comparing the predicted rate of cognitive decline to the actual rate of cognitive decline, where a deviation in the predicted versus actual rate of cognitive decline is indicative of the effectiveness of the AD therapeutic.

In yet another embodiment, the invention is a method for evaluating the relative effectiveness of multiple AD therapeutics comprising: (a) selecting a group of Alzheimer's disease patients; (b) conducting initial prognostic assessment of each AD patient, where the prognostic assessment comprises a cognitive assessment of the patient and a biomarker analysis of a fluid sample from the patient; (c) comparing the baseline CSF biomarker levels to a statistically significant slope (SSS) obtained from a standard AD patient panel; (d) determining the predicted rate of cognitive decline for each AD patient, where the predicted rate of cognitive decline is the predicted decrease in a CAMCOG or MMSE score; (e) dividing the selected AD patients of steps (a)-(d) into multiple groups; (f) administering an AD therapeutic to one of the subdivided groups of AD patients; (g) conducting one or more subsequent prognostic assessments on a periodic basis of each AD patient; (h) determining an actual rate of cognitive decline in the AD patient; and (i) comparing the predicted rate of cognitive decline to the actual rate of cognitive decline for each AD patient, where a deviation in the predicted versus actual rate of cognitive decline is indicative of the effectiveness of the AD therapeutic; and (j) determining the relative effectiveness of each AD therapeutic by comparing the deviation in the predicted versus actual rate of cognitive decline for each subgroup to the other.

### BRIEF DESCRIPTION OF THE DRAWINGS

FIGS. 1A-D show the group means, confidence intervals, and group distributions for the 1 and 2 year decline in CAMCOG (FIGS. 1A and 113, respectively) and MMSE (FIGS. 1C and 1D, respectively) scores in the AD and control groups. AD patients demonstrated significant decline compared to control subjects in both CAMCOG and MMSE scores at 1 year and 2 years (Wilcoxon p<0.0001 for all comparisons). CAMCOG and MMSE were generally well-correlated in this cohort.

FIGS. 2A-D show the graphical output of a resistant linear regression analysis of baseline age and 1 and 2 year decline in CAMCOG (FIGS. 2A and 213, respectively) and MMSE (FIGS. 2C and 2D, respectively) scores in the AD group. Slopes, confidence intervals, and p-values (t-test) are provided for the regression lines. Baseline age was not a statistically significant predictor of CAMCOG or MMSE decline in the AD group (A) at 1 or 2 years. Data from the control group (C) are also presented but were not included in the regression model.

FIGS. 3A-D show the graphical output of a resistant linear regression analysis of baseline score and 1 and 2 year decline in CAMCOG (FIGS. 3A and 3B, respectively) and MMSE scores (FIGS. 3C and 3D, respectively) in the AD group. Slopes, confidence intervals, and p-values (t-test) are provided for the regression lines. Baseline cognitive score was not a statistically significant predictor of CAMCOG or MMSE decline in the AD group (A) at 1 or 2 years. Data from the control group (C) are also presented but were not included in the regression model.

FIGS. 4A-D show the graphical output of a resistant linear regression analysis of baseline CSF Aβ42 and 1 and 2 year decline in CAMCOG (FIGS. 4A and 4B, respectively) and MMSE (FIGS. 4C and 4D, respectively) scores in the AD group. Slopes, confidence intervals, and p-values (t-test) are provided for the regression lines. Baseline CSF Aβ42 was not a statistically significant predictor of CAMCOG or MMSE decline in the AD group (A) at 1 or 2 years. Data from the control group (C) are also presented but were not included in the regression model.

FIGS. 5A-D show the graphical output of a resistant linear regression analysis of baseline CSF tau and 1 and 2 year decline in CAMCOG (FIGS. 5A and 5B, respectively) and MMSE (FIGS. 5C and 5D, respectively) scores in the AD group. Slopes, confidence intervals, and p-values (t-test) are provided for the regression lines. The relationship between higher baseline CSF tau and greater decline in both CAMCOG and MMSE in the AD group (A) was similar at 1 year and 2 years, and achieved statistical significance at 2 years. Data from the control group (C) are also presented but were not included in the regression model.

FIGS. 6A-D show the graphical output of a resistant linear regression analysis of baseline CSF tau/Aβ42 and annual decline in CAMCOG (FIGS. 6A and 6B, respectively) and MMSE (FIGS. 6C and 6D, respectively) scores in the AD group. Slopes, confidence intervals, and p-values (t-test) are provided for the regression lines. The relationship between higher baseline CSF tau/Aβ42 and greater decline in both CAMCOG and MMSE in the AD group (A) was similar at 1 year and 2 years, and achieved statistical significance for CAMCOG at both intervals and for MMSE at 2 years. Data from the control group (C) are also presented but were not included in the regression model.

FIGS. 7A and 7B show the graphical output of a power analysis demonstrating that baseline adjustment for CSF tau/Aβ42 in an AD population could potentially reduce the sample size required (maintaining 80% power) to observe a treatment effect on decline in CAMCOG (FIG. 7A) or MMSE (FIG. 7B) scores.

FIGS. 8A-8E show the graphical output of a non-linear mixed effects modeling of long-term (range 6 months-8 yrs) AD CAMCOG data from 5 individual patients. The modeled curves show the CAMCOG decline for each patient over 10 years. The horizontal lines represent baseline levels of tau and ptau-181, as labeled, for each patient. The modeling shows that patients with lower baseline levels of tau and ptau-181 demonstrate a more gradual CAMCOG decline over 10 years (e.g., FIG. 8A), whereas patients with higher baseline levels of tau and ptau-181 demonstrate a more rapid decline over the same period (e.g., FIG. 8E).

FIG. 9 shows the non-linear mixed effects curves fit for 39 patients included in the long-term analysis herein. Higher levels of baseline CSF tau are associated with faster CAMCOG decline. The mean time for CAMCOG to decline by 50% was reduced by approximately 50% for patients with high baseline tau (97.5% quantile, bottom curve) compared to those with low baseline tau (2.5% quantile, top curve). The mean time for CAMCOG decline for tau at the 50% quantile is shown by the middle curve.

### DETAILED DESCRIPTION OF THE INVENTION

### Definitions

As used herein, the term “biomarker” or “biochemical marker” refers to a protein that is to be analyzed biochemically and/or monitored over time, for example, Aβ42 or Tau.

As used herein, the term “prediction” or “prediction of cognitive decline” or “cognitive prediction” or “cognition prediction” refers to the translation or estimation of a cognitive score on a suitable scale from a set of biochemical markers, that is, to assign an equivalent cognitive score based on where they fit within the statistically relevant panel. This can be done for MMSE based on a scale of 0 to 30 and for CAMCOG based on a scale of 0 to 107.

As used herein, the term “monitoring Alzheimer's disease” means both the ability to classify a subject as AD or control as well as the ability to predict the cognitive status of the individual, including MMSE and total CAMCOG.

As used herein, the term “classifying the disease state” means that a subject is classified as either having the Alzheimer's disease or as being normal.

As used herein, the term “marker panel” refers to the biomarker panel consisting of CSF Aβ40, Aβ42, sAPPα, sAPPβ, tau, and ptau-181 as defined in the examples.

As used herein, the term “amyloid markers” refers to the biomarker panel consisting of CSF Aβ40, Aβ42, sAPPα, and sAPPβ as defined in the examples.

As used herein, the term “tau” refers to the total tau protein in a given sample or assay, regardless of phosphorylation state.

As used herein, the terms “ptau” and “ptau-181” refer to the subset of tau proteins which contain a phosphorylation site at a specified amino acid within the protein, in particular for the assays used herein, at amino acid position 181.

As used herein, the term “tau markers” refers to the biomarker panel consisting of CSF tau and ptau-181 as defined in the examples.

As used herein, the term “MMSE” refers to the Mini-Mental State Examination used in the cognitive assessment community.

As used herein, the term “total CAMCOG” or “CAMCOG” refers to the cognitive and self-contained part of the Cambridge Examination for Mental Disorders of the Elderly (CAMDEX) used in the cognitive assessment community.

As used herein, the term “CERAD” refers to the Consortium to Establish a Registry for Alzheimer's Disease used in the neuropathological community. As used herein, the term “CSF” refers to cerebrospinal fluid.

### Biomarker Studies

Biomarkers can be used to both define a disease state as well as to provide a means to predict physiological and clinical manifestations of a disease. Three commonly discussed ways in which biomarkers could be used clinically are: 1) to characterize a disease state, i.e. establish a diagnosis, 2) to demonstrate the progression of a disease, and 3) to predict the progression of a disease, i.e. establish a prognosis. Establishing putative biomarkers for such uses typically requires a statistical analysis of relative changes in biomarker expression either cross-sectionally and/or over time (longitudinally). For example, in a state biomarker analysis, levels of one or more biomarkers are measured cross-sectionally, e.g. in patients with disease and in normal control subjects, at one point in time and then related to the clinical status of the groups at the same point in time. Statistically significant differences in biomarker expression can be linked to presence or absence of disease, and would indicate that the biomarkers could subsequently be used to diagnose patients as either having disease or not having disease. In a progression analysis, levels of one or more biomarkers and clinical status are both measured longitudinally. Statistically significant changes over time in both biomarker expression and clinical status would indicate that the biomarkers under study could be used to monitor the progression of the disease. In a prognostic analysis, levels of one or more biomarkers are measured at one point in time and related to the change in clinical status from that point in time to another subsequent point in time. A statistical relationship between biomarker expression and subsequent change in clinical status would indicate that the biomarkers under study could be used to predict disease progression.

Results from prognostic analyses can also be used for disease staging and for monitoring the effects of drugs. The prediction of variable rates of decline for various groups of patients allows them to be identified as subgroups that are differentiated according to disease severity (i.e. less versus more) or stage (i.e. early versus late). Also, patients treated with a putative disease-modifying therapy may demonstrate an observed rate of cognitive decline that does not match the rate of decline predicted by the prognostic analysis. This could be considered evidence of drug efficacy.

The National Institute of Aging (NIA) consensus white paper on AD biomarkers (Regan Research Institute and NIA Consensus Report of the working group on ‘Molecular and Biochemical Markers of Alzheimer's Disease,’ reported at Neurobiology of Aging, 19(2): 109-116 (1998) (hereinafter “1998 NIA Consensus”) outlines several non-limiting uses of Alzheimer biomarkers. In particular, biomarkers of AD, either in the form of individual markers or multi-analyte panels can be used for multiple purposes: (1) to aid in the classification or diagnosis of the disease state of an individual to complement traditional clinical diagnosis with an objective measurement; (2) for epidemiological screening to select an enriched population or to characterize the prevalence of disease or demographics of any given epidemiological study; (3) for predictive testing or prognostic purposes of indicating who is susceptible to further neurodegenerative and cognitive decline; (4) for studying brain-behavior relationships; and (5) for monitoring disease progression or response to treatment in clinical trials and clinical practice. In practice the latter purpose has two separate aspects, including, (A) to determine whether a treatment induces a measurable biochemical change and (B) to determine whether treatment changes the progression of the illness, using the biomarker or multi-analyte panel as an index of disease status or state. The 1998 NIA Consensus also stated that a proposed biomarker or multi-analyte panels should include as many of the features of an ideal marker, including: (1) be able to detect a fundamental feature of AD neuropathology; (2) be validated in neuropathologically confirmed AD cases; (3) be precise (ability to detect AD early in its course and distinguish it from other dementias); (4) be reliable; (5) be non-invasive; (6) be simple to perform; and lastly (7) be inexpensive. It has been acknowledged and remains the case that no known biomarker for Alzheimer's meets the 1998 NIA criteria indicated.

The literature describes other multi-analyte type analyses that have been conducted. For example, WO 2004/104597, “Method for Prediction, Diagnosis, and Differential Diagnosis of AD” describes methods of predicting disease status via an x/y ratio of Aβ peptides. WO 2005/047484, “Biomarkers for Alzheimer's Disease” describes a series of markers that can be used for the assessment of disease state and other scientifically interesting avenues. WO 2005/052592, “Methods and Compositions for Diagnosis, Stratification, and Monitoring of Alzheimer's Disease and Other Neurological Disorders in Body Fluids” teaches methods and markers gleaned from plasma for the monitoring of Alzheimer's disease, WO 2006/009887, “Evaluation of a Treatment to Decrease the Risk of a Progressive Brain Disorder or to Slow Brain Aging” teaches methods and ways to use brain imaging to measure brain activity and/or structural changes to determine efficacy of putative treatments for brain-related disorders.

In order to develop new therapies to treat AD, clinical trials in AD patient populations must use cognitive testing to assess progression of the disease in order to determine whether the therapy under study has a positive effect on disease progression. However, the variability in patient response associated with cognitive testing, due to the progressive and variable course of the disease, is large enough to inhibit the ability of these tests to sensitively detect drug signals. Having a homogeneous patient population at the start of a clinical trial would minimize the “noise” introduced from the variance associated with AD. The ability to predict cognitive decline in AD patients over 1 to 2 years, the length of a typical AD clinical trial, or to stage AD patients, would greatly help to establish a more homogeneous clinical trial population at the start of the trial. This, in turn, could reduce subsequent variability and improve the chance to detect positive drug effects in AD clinical trials.

The variable nature of the progression of AD also presents a challenge in managing AD patients. A high versus a low rate of progression over the course of the illness ultimately determines how aggressively social support and medical intervention might be applied. The ability to predict the rate of long-term decline in AD could contribute to the ability to plan for various treatment contingencies.

### Clinical Diagnosis

For the methods described herein, a clinical diagnosis of Alzheimer's disease was made for each patient according to the criteria of the National Institute of Neurological and Communicative Disorders and Stroke and of the Alzheimer's Disease and Related Disorders Association (NINCDS-ADRDA). The criteria for a diagnosis of probable AD includes (1) dementia established by clinical examination and documented by MMSE or other similar examination and confirmed by neuropsychological testing; (2) deficits in two or more areas of cognition; (3) progressive worsening of memory and other cognitive functions; (4) no disturbance of consciousness; (5) onset between the ages 40 and 90, most often after age 65; and (6) absence of systemic disorders or other brain diseases that in and of themselves could account for the progressive deficits in memory and cognition.

A clinical diagnosis of an individual for AD or dementia would generally include some form of mental or cognitive assessment, which could be carried out by various methods including the Alzheimer's Disease Assessment Scale-Cognitive (ADAS-Cog), the Global Deterioration Scale (GDS), the Clinical Dementia Rating—summary of boxes (CDR-SB), the cognitive component of the Cambridge Mental Disorders of the Elderly Examination (CAMCOG), or more typically a Mini-Mental State Exam (MMSE). The CAMCOG is a small neuropsychological battery, with tests across multiple cognitive domains, that has a range in scores from 0 to 107. Patients with dementia typically score below 80 on the CAMCOG. (Roth M, et al. CAMDEX, “A standardised instrument for the diagnosis of mental disorder in the elderly with special reference to the early detection of dementia,” Br. J. Psychiatry, 149: 698-709, 1986; Lolk, A., et al., “CAMCOG as a screening instrument for dementia: the Odense Study,” Acta Psychiatr. Scand., 102:331-335, 2000). CAMCOG results generally correlate well with MMSE, though the tests differ in some psychometric properties. MMSE scores have a maximum of 30, with scores generally classified as mild (21-26), moderate (15-20) and severe (14 or less). Scores for ADAS-Cog range from 0 (best possible) to 70 (worse possible), with scores of around 23 being the cutoff for mild impairment and scores of about 35 or higher correlating with moderate and above impairment. Scores for CDR have a maximum of 4, with scores classified as normal (0), mild (0.5-1), moderate (2), and severe (3-4). Similarly, scores for GDS range from stage 1 (best) to stage 7 (worst), with grade 4 being comparable to and ADAS-Cog score of about 22.5 for mild impairment and stage 5 being comparable to an ADAS-Cog score of about 35 for moderate impairment. See Folstein et al., J. Psychiat. Res., 12: 189-198, 1975, for a general discussion of MMSE in relationship to AD and dementia. See Doraiswamy et al., Neurology, 48 (6): 1511-1517, 1997, for a comparison of ADAS-Cog, MMSE and GDS scoring and validity. ADAS-Cog and MMSE have been generally acceptable for use in assessment of efficacy in clinical trials.

### Methods of Cognitive Prognosis

Applicants herein have developed a method to predict the future clinical state, as assessed by cognitive endpoints, in Alzheimer's disease patients. The method comprises the identification and analysis of statistically relevant biomarkers and biomarker ratios in a patient fluid sample, such as CSF, through the use of linear regression analysis or non-linear mixed effects modeling. The method of the invention herein more accurately and objectively assesses the status of an individual for the purposes of disease classification and predicting cognitive endpoints, such as MMSE and CAMCOG.

Those of ordinary skill in the art would recognize and appreciate that CSF levels of amyloid beta and tau-related proteins, as a reflection of the ongoing pathologic processes in AD, at any given time might be used to predict the future course of the disease. Applicants herein have shown that a baseline evaluation of CSF biomarkers can be used to predict subsequent cognitive decline. In patients with memory impairments who are followed for several years, levels of CSF amyloid markers (Aβ42, Aβ40, sAPPα, sAPPβ) and tau markers (tau, ptau-181) were found to predict the cognitive decline in AD patients.

The invention claimed herein is a methodology that can be used to predict the average cognitive decline in patients with Alzheimer's disease using CSF biomarkers. Analysis of CSF for expression of amyloid markers (Aβ40, Aβ42, sAPPα, sAPPβ) and tau markers (tau, ptau-181) was performed at a baseline time point. Levels of these markers were then assessed in a linear regression analysis to predict decline in the CAMCOG and MMSE cognitive tests over a short-term period of 1-2 years. Levels of these markers were also assessed in a non-linear mixed effects model to predict decline in the CAMCOG over a long-term period of a decade.

In the present method, a community sample of AD patients and healthy subjects, as controls, was recruited into the OPTIMA cohort. CSF specimens were collected at a baseline visit from 48 patients with a clinical diagnosis of AD according to NINCDS-ADRDA criteria, 38 of whom had pathologically-confirmed diagnoses, and from 89 age-matched healthy subjects. The demographic characteristics of this population are described in Table 1. CSF specimens were analyzed for levels of amyloid (Aβ40, Aβ42, sAPPα, sAPPβ) and tau (tau, ptau-181) markers. Those of ordinary skill in the art would understand that each biomarker assay would have needed to undergo fit-for-purpose assay validation, including assessment of key issues such as freeze-thaw stability, dilution linearity, precision, and sensitivity.

TABLE 1

| | |
|---|---|
| CTL | AD |
| (N = 89) | (N = 48^{a}) |

Gender

| | | |
|---|---|---|
| Female | 46(52%) | 29(60%) |
| Male | 43(48%) | 19(40%) |

Age (years)

| | | |
|---|---|---|
| Mean (SD) | 69.9(10.5) | 69.5(8.7) |
| Range | 36-94 | 53-83 |

MMSE

| | | |
|---|---|---|
| Mean (SD) | 28.6(1.6) | 18(4.9) |
| Range | 24-30 | 10-28 |

CAMCOG

| | | |
|---|---|---|
| Mean (SD) | 98.8(4.6) | 62(15) |
| Range | 86-106 | 28-93 |

ApoE Genotype

| | | |
|---|---|---|
| E4(−) | 61(69%) | 16(33%) |
| E4(+) | 28(31%) | 32(67%) |

^{a}38 patients were pathologically confirmed CTL = control; AD = Alzheimer's disease; N = Number of subjects/patients; SD = Standard deviation; MMSE = Mini-mental state examination; CAMCOG = Cambridge cognitive examination; ApoE = Apolipoprotein E

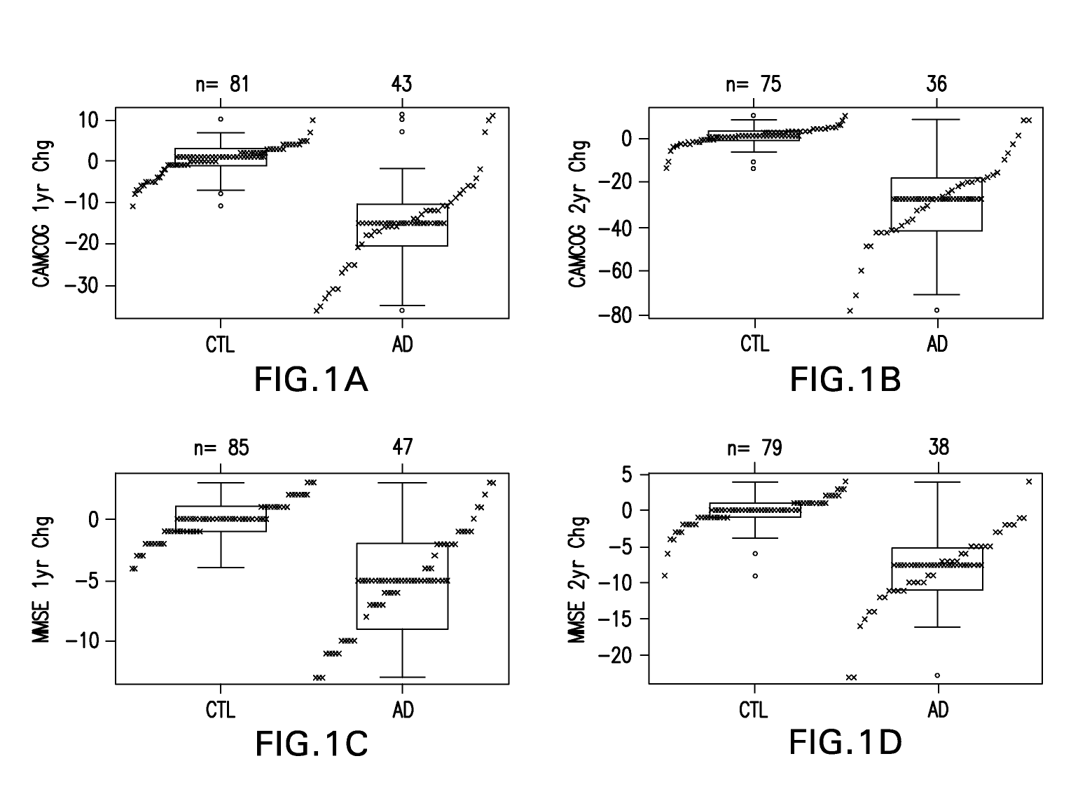

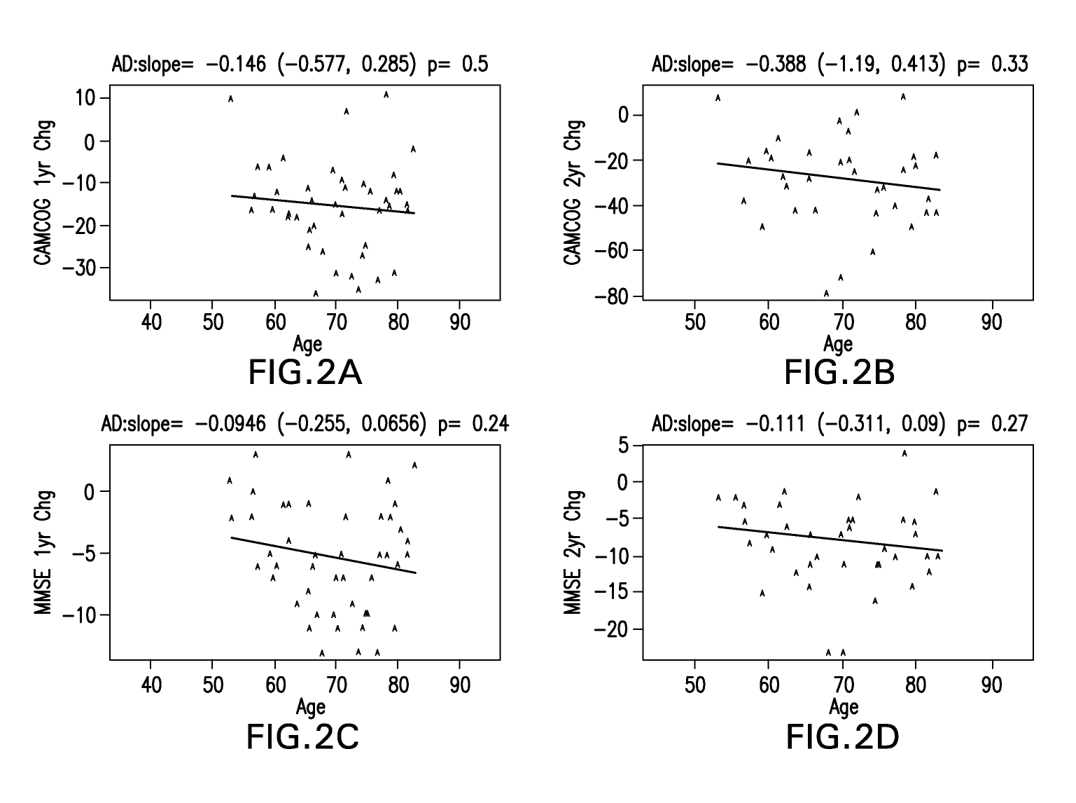

The AD patients and control subjects were also cognitively assessed on a yearly basis following the baseline visit using MMSE and CAMCOG. Compared to control subjects, AD patients demonstrated significant declines in CAMCOG scores at 1 year (FIG. 1A) and 2 years (FIG. 1B) from baseline and in MMSE scores at 1 year (FIG. 1C) and 2 years (FIG. 1D) from baseline.

A statistical model was constructed, using linear regression analysis, to describe in AD patients (“A” in FIGS. 2-6) the relationship between several different variables and short-term change in MMSE and CAMCOG scores over 1 to 2 years. This relationship is represented by the regression line. A horizontal regression line (slope=0) indicates that there is no relationship between two variables under study because as one variable changes, the other remains constant. A regression line with either a positive or negative slope, that is, one that is statistically different from horizontal (p≦0.05 level), indicates that a relationship between two variables exists because both are changing concurrently, referred to herein as a statistically significant slope (SSS). Consequently, the position of one variable on the regression line can be used to determine the value of the second variable even if the second variable is not directly measured (i.e. the value of the second variable can be predicted from measurement of the first variable). Control subjects (“C” in FIGS. 2-6) were not used in the regression modeling because they did not demonstrate significant decline in either MMSE or CAMCOG scores at 1 year or 2 years after baseline (FIGS. 1A-1D). However, control data points are provided on all regression plots for comparison purposes.

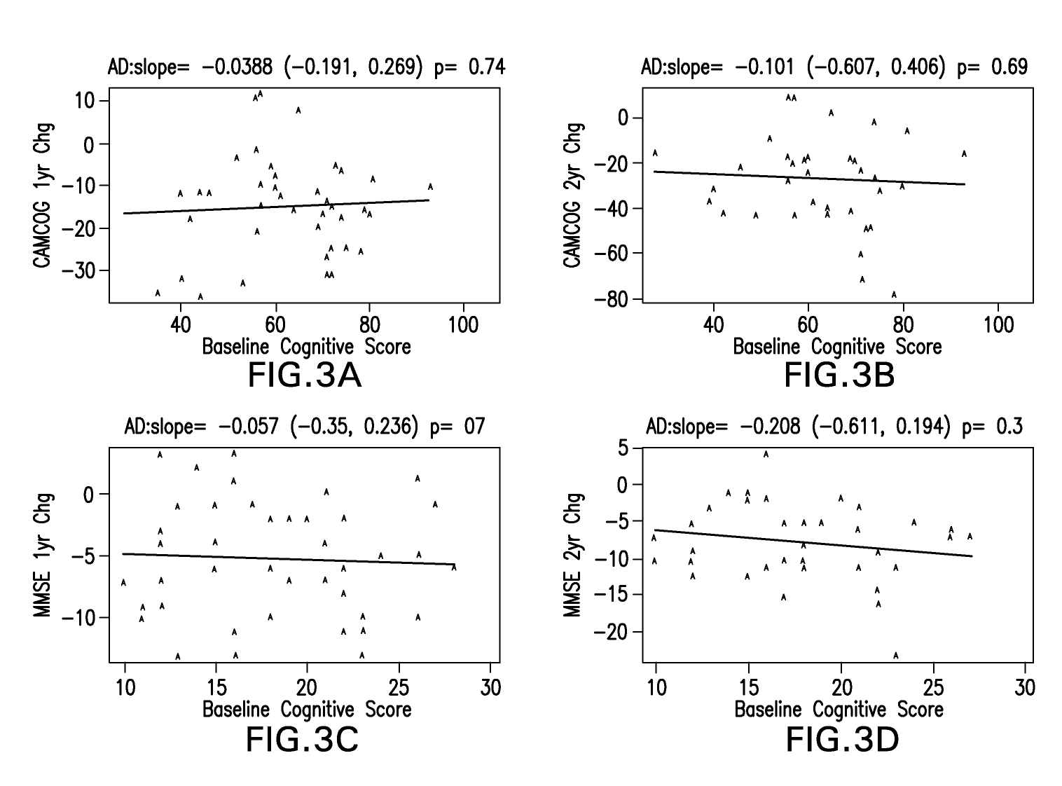

Regression analysis was used to describe the relationship between baseline demographic variables (age and baseline cognitive score) and short-term change in MMSE and CAMCOG scores in AD patients. None of the regression curves describing the relationship between patient age and CAMCOG score (FIGS. 2A and 2B) or MMSE score (FIGS. 2C and 2D) decline at 1 year and 2 years, respectively, had a statistically significant slope (SSS). Similarly, none of the regression curves describing the relationship between baseline cognitive score and CAMCOG score (FIGS. 3A and 3B) or MMSE score (FIGS. 3C and 3D) decline at 1 year and 2 years, respectively, had a significant slope. Consequently, neither patient age at baseline nor baseline cognitive scores were significant predictors of subsequent cognitive decline in AD patients.

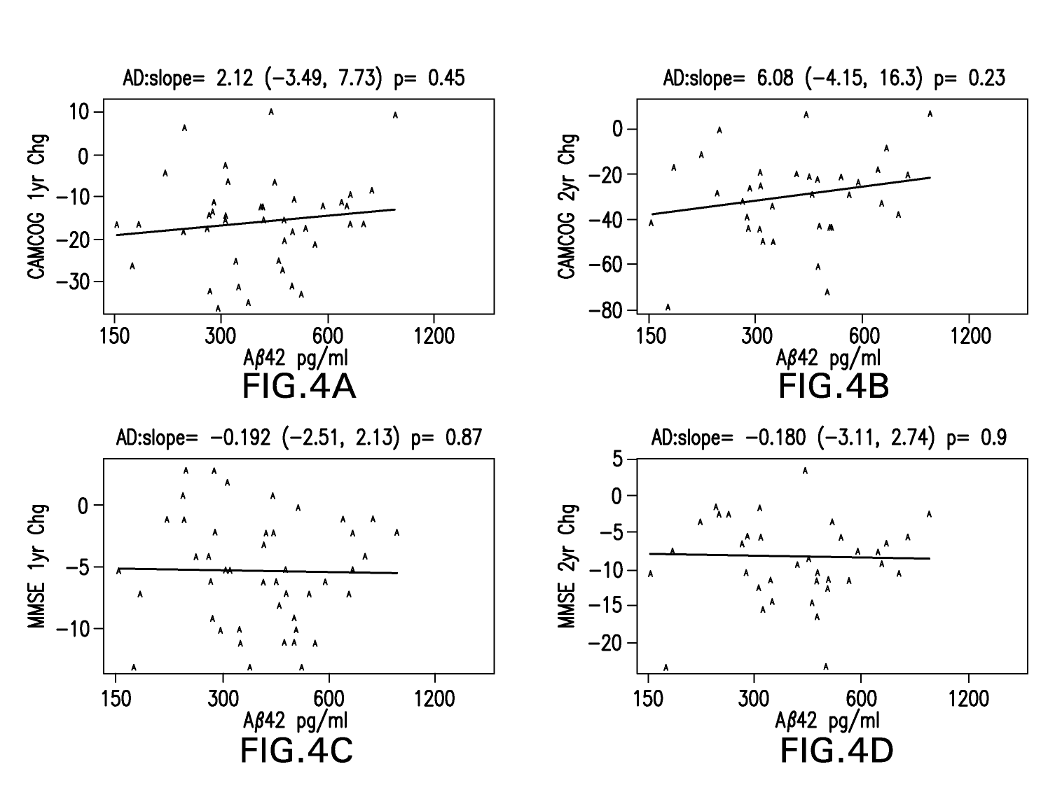

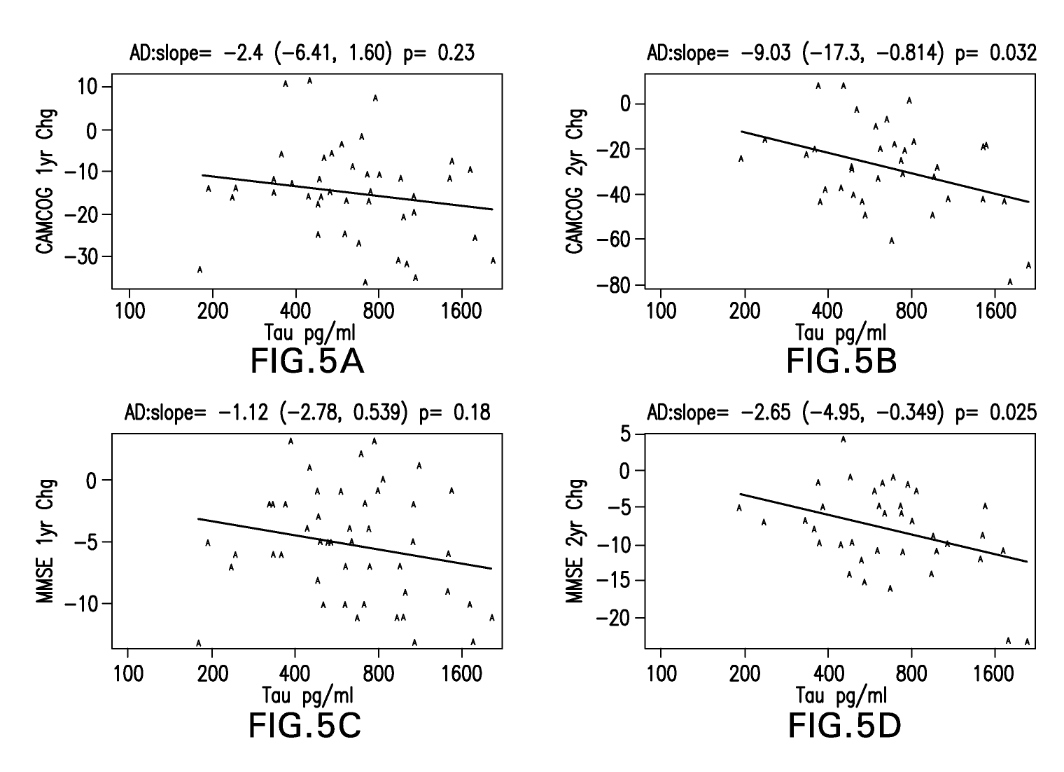

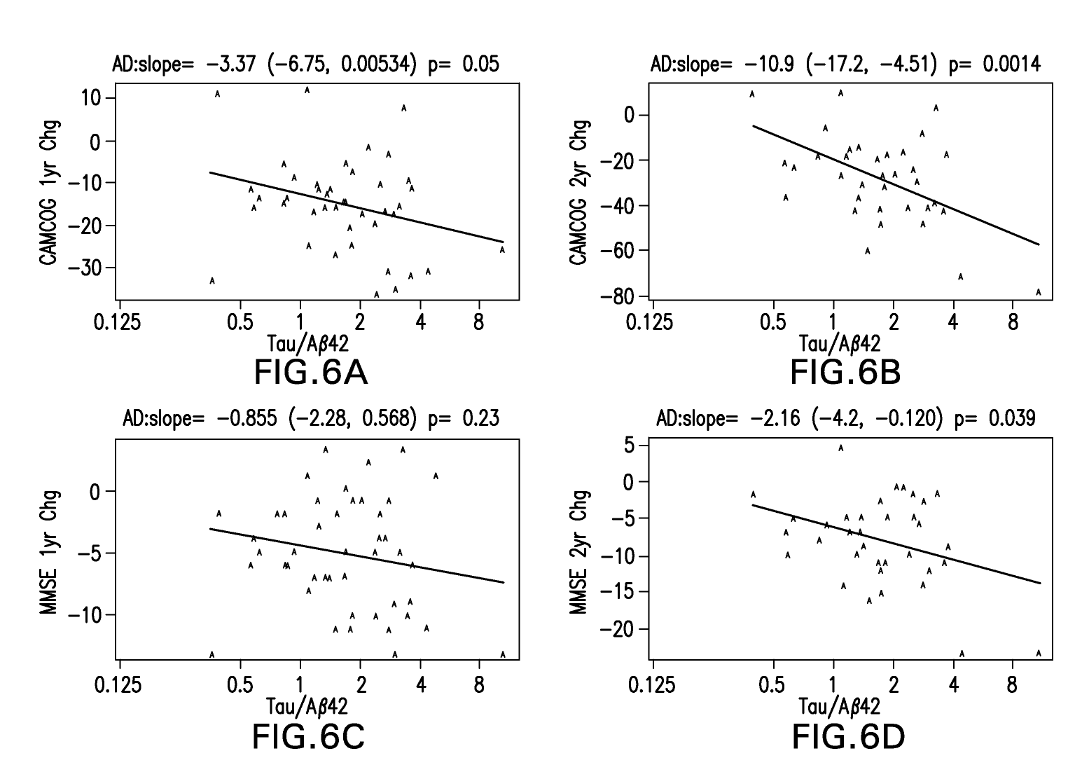

Regression analysis was also used to describe the relationship between baseline levels of CSF markers and short-term change in MMSE and CAMCOG in AD patients. None of the regression curves describing the relationship between baseline levels of CSF Aβ42 and CAMCOG score (FIGS. 4A and 4B) or MMSE score (FIGS. 4C and 4D) decline at 1 year and 2 years had a statistically significant slope. Similar results (data not shown) were obtained with other amyloid markers (Aβ40, sAPPα, sAPPβ). Regression curves describing the relationship between baseline levels of CSF tau and CAMCOG score (FIG. 5A) or MMSE score (FIG. 5C) decline at 1 year did not have statistically significant slopes, but regression curves describing CAMCOG score (FIG. 5B) or MMSE (FIG. 5D) decline at 2 years were significant. Similar results were obtained with CSF ptau-181 (data not shown). All of the regression curves describing the relationship between a ratio of baseline CSF tau/Aβ42 and CAMCOG score (FIGS. 6A and 6B) or MMSE score (FIGS. 6C and 6D) decline at 1 year and 2 years had statistically significant slopes. Similar results were obtained with other tau to amyloid marker ratios (data not shown). Taken together, Applicants found that baseline levels of CSF amyloid markers were not significant predictors of subsequent cognitive decline in AD patients. Conversely, baseline levels of CSF tau markers were significant predictors of cognitive decline at the 2-year time point. Ratios of baseline levels of CSF tau/amyloid were significant predictors of cognitive decline at both the 1 year and 2 year mark.

It should be noted that the output (AD standard curve) from the regression analysis, i.e. the plots showing the SSS (FIGS. 2-6), of the above described AD patients (AD standard panel) would form the standard curves for the specified CSF marker and would be used to predict the short-term rate of cognitive decline, that is the point decrease for a CAMCOG or MMSE score, for an unknown AD patient having the same baseline level of the CSF marker. For example, a group of AD patients have a CSF baseline level of tau/Aβ42 ratio of 1 would have a predicted rate of cognitive decline of 10 points over 1 year or 20 points over 2 year for CAMCOG (FIGS. 6A and 6B, respectively).

A separate statistical model was constructed, using non-linear mixed effects modeling, to describe the relationship between baseline levels of amyloid and tau markers and change in CAMCOG scores over a period of about 10 years. This relationship, represented by the mixed effects model curve, allows for long-term prediction of CAMCOG score decline over 10 years in subsequent patients for whom baseline levels of CSF biomarkers are assessed.

It should be noted that while Applicants have shown a relationship between baseline levels of specific tau and amyloid markers, those of ordinary skill in the art would understand and appreciate that other CSF markers can be measured and analyzed in a similar manner. Those markers having a relationship between baseline levels and short-term or long-term change in CAMCOG and MMSE scores, as evidenced by a regression line having a SSS, can be employed in the methods described herein.

A power analysis was performed in order to determine whether baseline assessment of CSF markers would allow for a reduction in the number of patients required to demonstrate a significant change in cognitive performance at 1 year and 2 years from baseline. A power analysis is used as an indicator of sensitivity because an increase in the sensitivity of an endpoint is associated with a reduced sample size required to demonstrate change in that endpoint. A power curve describes a relationship between the number of patients required to have a certain percentage power to detect change in an endpoint (commonly, 80% for clinical trials) versus change in the same endpoint. A shift downward in a power curve, adjusted for a variable of interest, would indicate that following adjustment for that variable, fewer patients would be required to observe endpoint changes at the same power level (i.e. the level of sensitivity of the endpoint is increased by the adjustment). See, Thal, L. et al., “The Role of Biomarkers in Clinical Trials for Alzheimer Disease,” Alzheimer Dis. Assoc. Disord., 20(1):6-15, January-March 2006.

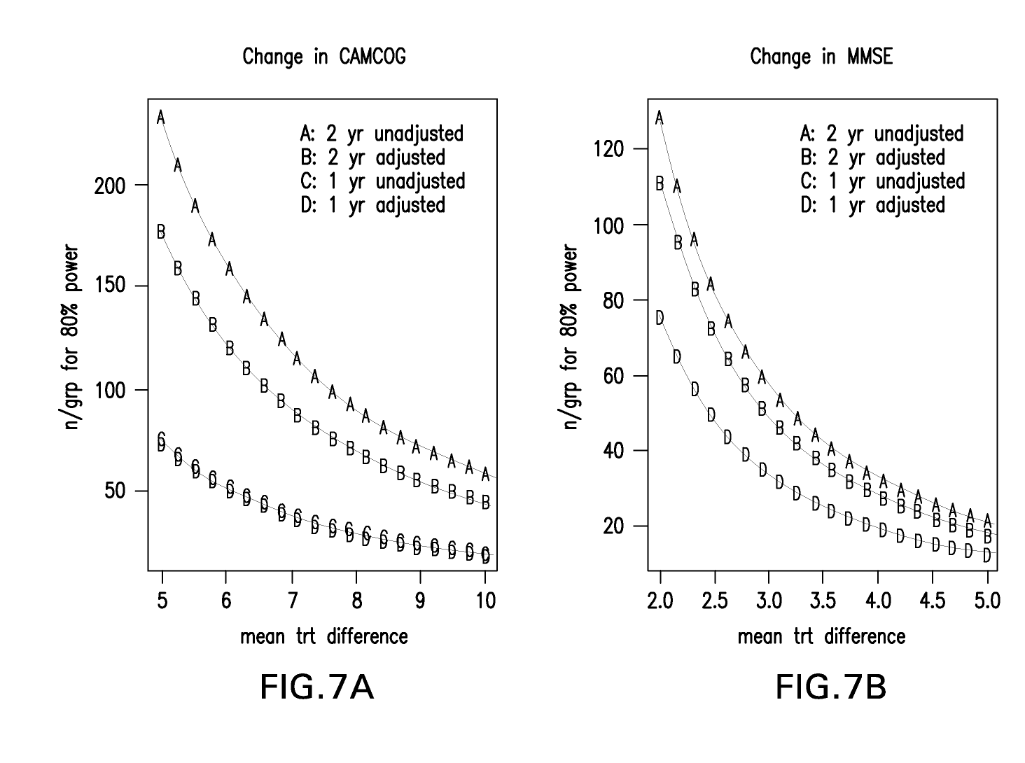

The power analysis herein was used to describe the relationship between the number of AD patients required to observe a change in CAMCOG scores at 80% power versus statistical change in CAMCOG scores at 1 year (FIG. 7A, curve C) and 2 years (FIG. 7A, curve A). Adjustment for levels of baseline CSF tau/Aβ at 1 year (FIG. 7A, curve D) and at 2 years (FIG. 7A, curve B) demonstrated a downward shift in the adjusted power curve at 2 years, indicating an increased sensitivity to detect change in CAMCOG score following the adjustment. Similar results were obtained by adjusting for other CSF tau/amyloid ratios.

Similarly, the power analysis was used to describe the relationship between the number of AD patients required to observe change in MMSE scores at 80% power versus statistical change in MMSE scores at 1 year (FIG. 7B, curve C) and 2 years (FIG. 7B, curve A). Adjustment for levels of baseline CSF tau/Aβ at 1 year (FIG. 7B, curve D) and at 2 years (FIG. 7B, curve B) demonstrated a downward shift in the adjusted power curve at 2 years, indicating an increased sensitivity to detect change in MMSE score following the adjustment. Similar results were obtained by adjusting for other CSF tau/amyloid ratios.

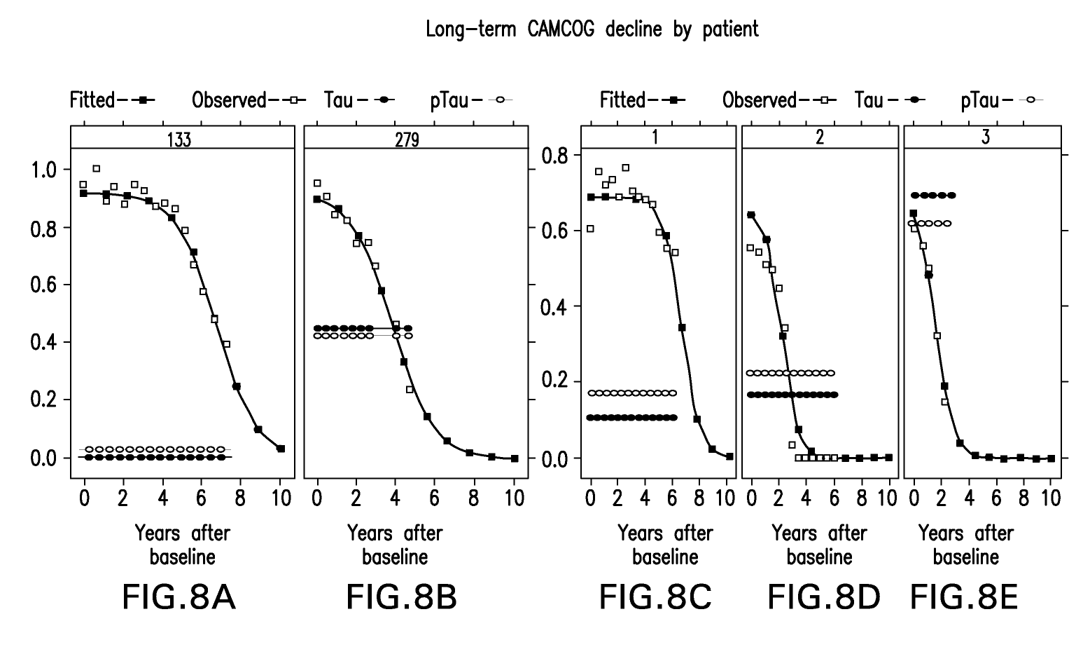

A separate statistical model was constructed, using a non-linear mixed effects modeling, to describe the relationship in AD patients between CAMCOG scores and time from baseline over a long-term period of about 10 years. This relationship is represented by a mixed effects model curve and indicates that the long-term decline in AD cognition over this period is non-linear. Modeling of curves for five individual patients for whom baseline levels of tau and ptau-181 were assessed (FIGS. 8A-8E) was performed. In these plots, the dots represent observed CAMCOG scores and the curves represent the modeled CAMCOG decline, The horizontal lines represent baseline levels of tau and ptau-181 as labeled. This modeling indicates that patients with lower baseline levels of tau and ptau-181 demonstrate more gradual CAMCOG decline with a longer plateau phase (FIGS. 8A and 8C) whereas patients with higher baseline levels of tau and ptau-181 demonstrate a more rapid CAMCOG decline over the same period (FIGS. 8B and 8E).

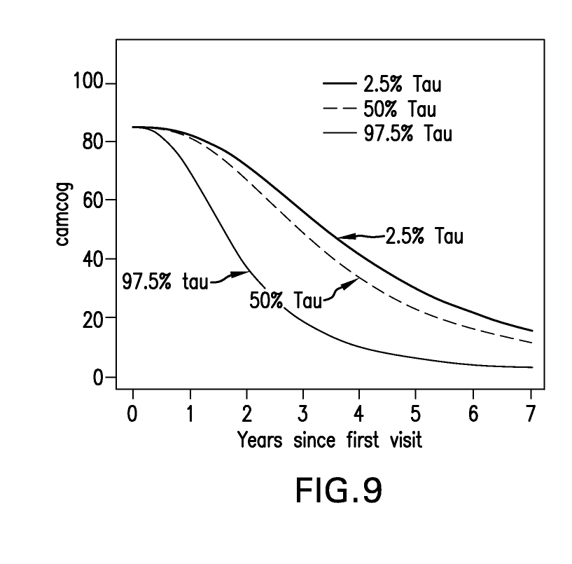

Average non-linear mixed effects curves were fit for 39 AD patients included in the long-term analysis to demonstrate the impact of levels of baseline CSF tau on CAMCOG score decline over about 10 years (FIG. 9). The mean time for CAMCOG scores to decline by 50% was reduced by approximately 50% for patients with high baseline levels of tau (97.5% quantile, bottom curve) compared to those with low baseline levels of tau (2.5% quantile, top curve). CAMCOG score decline for patients with intermediate baseline levels of tau at the 50% quantile is shown by the middle curve. These results show that higher levels of baseline CSF tau are associated with faster long-term CAMCOG decline. Although similar results were observed with ptau-181, baseline demographic variables and baseline levels of CSF amyloid markers were not significantly associated with CAMCOG decline in this model.

### Short Term Cognitive Prognosis

Applicants herein have shown through linear regression analysis that baseline levels of CSF amyloid and tau markers are related to subsequent cognitive decline in AD patients in the short term, defined herein as a period of 1 to 2 years from a baseline point in time. Applicants believe that this is the first study to demonstrate a relationship between levels of CSF biomarkers and subsequent short-term cognitive decline in AD patients. In particular, baseline levels of CSF tau or ptau-181 and ratios of baseline tau/amyloid were significantly related to subsequent decline. Consequently, for AD patients who have had baseline levels of these markers determined, the regression analyses herein can be used to predict short-term cognitive decline, i.e. rate of short term cognitive decline, in advance of actual observation and measurement of any decline. For example, baseline analysis of CSF tau/Aβ42 levels in a newly diagnosed group of AD patients can be related to an average decline in MMSE scores over 1 year from the appropriate regression plot (in this case, FIG. 6C). As such, the decline in MMSE score can be predicted for unknown group of AD patients in advance of MMSE assessment after 1 year of real-time clinical follow up. This represents a significant improvement over the current practice of using demographic variables to estimate AD cognitive decline in that it uses statistically significant differentiators to predict subsequent decline.

### Long Term Cognitive Prognosis

In another embodiment of the invention, Applicants have shown through non-linear effects modeling that baseline levels of CSF tau markers are related to subsequent cognitive decline in AD patients in the long term, defined herein as a period of about 10 years from a baseline point in time. Applicants believe that this is the first study to demonstrate a relationship between levels of CSF biomarkers and subsequent long-term cognitive decline in AD patients. In particular, baseline levels of CSF tau or ptau-181 were significantly related to subsequent long term decline. Consequently, for AD patients who have had baseline levels of these markers determined, the present method utilizing a mixed effects model can be used to predict long-term cognitive decline, i.e. rate of long term cognitive decline, in advance of actual observation and measurement of any decline. For example, baseline analysis of CSF tau levels in a newly diagnosed group of AD patients can be related to an average decline in CAMCOG scores over 10 years using the average mixed effects plot (FIG. 9). As such, the decline in CAMCOG scores can be predicted for the unknown group of AD patients in advance of CAMCOG assessment after 1 year of real-time clinical follow up. Currently, there is no accepted method for predicting long term AD cognitive decline.

The use of this long-term prognosis can assist caregivers in planning for long-term treatment contingencies due to the wide variability of long-term progression in AD patients. A high versus a low rate of long-term progression over the course of the illness could be used to determine how aggressively social support and medical intervention might be applied. The ability to predict rates of long-term decline would also allow for resource and treatment allocation well in advance of actual patient progression.

### Cognitive Prognosis in the Stratification of Patient Populations

Those of ordinary skill in the art will understand and appreciate that the inventive methodology described herein for predicting the short-term and long-term and long term rates of cognitive decline can be employed to stratify and AD patient populations for the purpose of conducting clinical trials and staging treatment. The ability to predict variable rates of decline for AD patients would allow clinicians to identify and select subgroups of patients for any given clinical trial. Potential clinical patients can be selected on the basis of baseline CSF Levels, for example, groups of patients having baseline levels of tau/Aβ42 of 1 or 2, FIGS. 6A-6D, or disease severity, either more versus less or stage (early versus late), as reflected in the rate of cognitive decline, the statistically significant slope (SSS), to provide a more homogeneous patient population. Similarly, one could use the rates of decline to stage treatment options, as to when to administer a given therapeutic agent.

### Cognitive Prognosis in the Evaluation of Drug Efficacy

In another embodiment of the invention, those of ordinary skill in the art will understand and appreciate that the inventive methodology described herein for predicting the short-term and long-term and long term rates of cognitive decline can be employed in the evaluation of drug efficacy. The present methodology can be employed as an endpoint surrogate to improve evaluations of drug efficacy and to increase the sensitivity of clinical, i.e. cognitive, endpoints in AD clinical trials. Drug efficacy could be defined not only by deviation from predicted rates of cognitive decline, but also the relative rates of deviation where one drug could be differentiated from another by the differences in deviation across the therapeutic agents.

In that an endpoint for any AD therapeutic assessment can be limited or obscured by the heterogeneity in the AD patient population due to varying states of disease and rates of progression, using the cognitive prognosis for the identification and selection of clinical trial patients who are similarly situated, i.e. exhibiting similar rates of cognitive decline, creates a more homogeneous study population, which in turn improves the sensitivity of the endpoint by greatly reducing or eliminating background noise resulting from progressive disease presentation, which in turns provides a better evaluation of the effect of the administered therapeutic, By way of example, using patients having baseline CSF tau/Aβ42 ratio of 1 (FIG. 6A-6D), one would predict a 10 point decline over 1 year or a 20 point decline over 2 years in CAMCOG scores (FIGS. 6A and 6B). After administration of a candidate therapeutic agent and subsequent periodic assessment, a deviation in the actual rate versus predicted rate of cognitive decline of CAMCOG scores from those receiving the candidate agent would be attributable to therapeutic effect. Similarly, a long-term prognosis could be used in a comparable manner where a deviation of actual long-term rate versus the predicted long-term rate of cognitive decline would be evidence of drug efficacy.

By extension, those of skill in the art would recognize that the amount of deviation from actual versus predicted rates of cognitive decline across groups of clinical patients receiving different candidate therapeutic agents could be used to determine the relative efficacy of different candidate agents. For example, as above, patients having baseline CSF tau/aβ42 ratio of 1 (FIGS. 6A-6D) can be identified and selected for a clinical trial of more than one candidate agent. The identified patient group would be further divided into two groups, those receiving candidate agent A (group A) and those receiving candidate agent B (group B). After administration of the candidate agents and subsequent periodic assessment, a comparison of the actual versus predicted rates of cognitive decline for group A and group B, and across the groups, would not only indicate the efficacy of each agent, but the relative efficacy versus the other candidate agent. A deviation of actual versus predicted rate of cognitive decline for one group that was greater than the deviation for the other group, or a deviation in actual versus predicted rates of cognitive decline in one group and none in the other, would be indicative of the relative efficacy of the candidate agents.

Moreover, those of skill in the art would understand and appreciate that the cognitive prognosis can be employed to increase the efficiency of clinical trials by allowing for a reduction in study sample size as shown by the power analysis included herein demonstrating that a reduced number of patients would be required to observe changes in cognitive scores over 1 to 2 years, a typical length for an AD clinical trial, following adjustment for baseline CSF levels of tau/Aβ42 (FIGS. 7A and 7B). Fewer numbers of clinical patients would translate to lower costs of trials, in addition to the efficiencies provided by a more homogeneous patient population in terms of endpoints and perhaps shorter duration of trials.

Those of skill in the art would recognize that this method of evaluating drug efficacy would not only be applicable to a clinical setting, but could be utilized for monitoring the effectiveness of any approved drug during or after introduction to the market. The ability to monitor the drug's effectiveness could also be used to modify treatment regiments and dosage amounts according to the patients' rate of cognitive decline.

### Example 1

### Selection of Patients and CSF Samples

OPTIMA (Oxford Project To Investigate Memory and Ageing) is a highly defined longitudinal cohort of community volunteers with interest in the periodic assessment of their memory and cognitive status who have been studied serially since 1988 and includes controls, AD and other dementias. There are over four hundred subjects and over 300 controls that undergo neuropsychological tests, CT and SPECT scans and various biochemical tests on their blood at regular intervals. Cerebrospinal fluid is obtained from a subset of patients who have consented specifically for this procedure. After death, autopsy is performed and the brains are examined by a neuropathologist to define brain pathology. To date, the autopsy rate has been 94%. All of the information and samples of the OPTIMA cohort are stored at the Radcliff Infirmary in Oxford, UK.

Applicants have analyzed for biomarker expression CSF specimens obtained ante-mortem from 48 subjects, 38 pathologically confirmed amyloid AD, and 89 clinical controls. The demographic characteristics of the pilot population at the time of CSF collection are shown in the Table 1. The AD and control groups were similar in age and gender distribution.

### Example 2

### Aβ40 Expression

Aβ40 was measured in the CSF with a human Aβ 1-40 Colorimetric solid phase sandwich Enzyme Linked Immuno-Sorbent Assay (ELISA) kit (catalogue #KHB3482, BioSource International, Camarillo, Calif.) following the manufacturer's recommendations. A standard sandwich immunoassay was performed wherein the analyte, Aβ40, was first captured with an antibody specific for the N-terminal half of Aβ and then detected with a second detection antibody specific for the Aβ40 neo-epitope. This sandwich immunoassay can be performed using any suitable antibody pair that measures Aβ40 or its truncated equivalents. The detection antibody consisted of rabbit anti-Aβ40 and a secondary anti-rabbit IgG:horse radish peroxidase (HRP) conjugate. HRP catalyzes the formation of a chromophore, tetramethylbenzidine (TMB), which was quantitatively measured at 450 nm to provide readout of Aβ40 concentration. This procedure was carried out according to the BioSource kit instructions. A blocking buffer was used to minimize non-specific interactions. Standards were used as received in the kit. Determinations of unknowns were made using a four parameter logistic fit to the standards measured in duplicate wells. Quality controls samples (low, mid, and high) were run on all plates to insure valid results consistent with previous measurements.

### Example 3

Aβ42 Expression

Aβ42 was measured with Innotest™ Aβ42 ELISA kit (Innogenetics Inc., Cat. #80040, Ghent, Belgium) following the manufacturer's recommendations with modifications as follows. Similar to the Aβ40 assay above, a standard sandwich immunoassay was performed wherein the analyte, Aβ42, was first captured with an antibody specific for the N-terminal half of Aβ (3D6) and then detected with a second detection antibody (21F12) specific for the Aβ42 neo-epitope. The assay utilized a mouse monoclonal capture antibody specific for the C-terminus of Aβ42. The detection system employed an N-terminal specific biotinylated mouse monoclonal antibody and a secondary conjugate made of horse radish peroxidase (HRP) labeled strepavidin. The HRP was used to convert tetramethyl benzidine to a chromophore which was quantitatively measured at 450 nm to provide readout of Aβ42 concentration. This sandwich immunoassay can be performed using any suitable antibody pair that measures Aβ42 or its truncated equivalents. A blocking buffer was used to minimize non-specific interactions. After detection of the amount of bound detection antibody with a substrate for a conjugated enzyme to the detection antibody, the amount of analyte was determined against a standard curve generated from a known master stock. In an attempt to reduce variability between kit lot numbers, Applicants deviated from the standard manufacturer's protocol by creating a concentrated solution of amino acid analyzed Aβ42 (0.778 mg/mL in DMSO). This was used across different kit lots instead of the standard material supplied by the manufacturer. The range of standards used for sample analysis was 5.45 to 350 pg/mL, Quality controls samples (low, mid, and high) were run on all plates to insure valid results consistent with previous measurements.

### Example 4

sAPPα, and sAPPβ Expression

When APP is processed by either α-secretase or β-secretase, it is cleaved into two fragments, of which the amino terminal fragment has been called the secreted APPα or β fragment. These two cleavage products of APP, sAPPα and sAPPβ, were measured with the MSD® sAPPα/sAPPβ Multiplex kit (MesoScale Discovery Cat #N41CB-1, Gaithersburg, Md.), following the manufacturer's recommendations. Unlike the previous Examples, this assay was run in a duplex format whereby two signals were read from a single well of a 96 well plate, enabling simultaneous determinations of both sAPPα and sAPPβ. In short, a standard sandwich immunoassay was performed wherein the analyte, now either of the sAPPs present in a human CSF samples, was first captured with an antibody specific a c-terminal region of sAPPα or the sAPPβ C-terminal neo-epitope, and then detected with a second detection antibody, in this case directed towards an n-terminal region of APP. This sandwich immunoassay can be performed using any suitable antibody pair that measures these analytes specifically. However, Applicants have assessed several antibodies in the literature and found that most have poor immunoreactivity to the naturally occurring isoforms and post-translational modifications of sAPP found in human CSF. A blocking buffer was used to minimize non-specific interactions. After detection of the amount of bound detection antibody, using the MSD TPA buffer solution as a substrate for a Ruthenium conjugated enzyme as detection antibody, one determined the amount of analyte against a standard curve generated from a known master stock. Quality controls samples (low, mid, and high) were run on all plates to insure valid results consistent with previous measurements.

### Example 5

t-tau Expression

Total tau (t-tau) expression was measured with a human tau (hTAU AG Innotest™) ELISA kit (Innogenetics Inc., catalogue number 80226, Ghent, Belgium) following the manufacturer's recommendations. Similar to the Aβ assays in Examples 2 and 3 above, a standard sandwich immunoassay was performed wherein the analyte, total tau protein independent of phoshorylation state, was first captured with a monoclonal antibody specific for all isoforms of tau and then subsequently bound by two biotinylated tau-specific antibodies. The final detection was performed by peroxidase-labeled streptavidin. This sandwich immunoassay can be performed using any suitable antibody pair that measures all tau species, including truncated equivalents. A blocking buffer was used to minimize non-specific interactions. After detection of the amount of bound detection antibody with a substrate for a conjugated enzyme to the detection antibody the amount of analyte was determined against a standard curve generated from a known master stock Quality control samples (low, mid, and high) were run on all plates to insure valid results consistent with previous total tau measurements.

### Example 6

ptau-181 Expression

Phosphorylated tau-181 (ptau-181) was measured with the Phospho-TAU (181P) Innotest™ ELISA kit (Innogenetics Inc., catalogue number 80062, Ghent, Belgium), following the manufacturer's recommendations. Similar to the total tau assay above, a standard sandwich immuno-assay was performed wherein the analyte, now tau protein phosphorylated at amino acid 181, was first captured with an antibody specific for all isoforms of tau and then detected with a second detection antibody which specifically detected tau molecules phosphorylated at threonine 181 (phosphotau-181). This sandwich immunoassay can be performed using any suitable antibody pair that measures specific phospho-181 tau species, including truncated equivalents. A blocking buffer was used to minimize non-specific interactions. After detection of the amount of bound detection antibody, typically with a substrate for a conjugated enzyme to the detection antibody, one determined the amount of analyte against a standard curve generated from a known master stock. Quality controls samples (low, mid, and high) were run on all plates to insure valid results consistent with previous total tau measurements.

### Example 7

### Statistical Prediction of Change in Cognitive Score Based on a Biomarker.

The prediction of one and two year change in cognitive score based on a biomarker was determined using a linear statistical model. The specific model used employed a Tukey bi-weight function (Venables, W. N. and Ripley, B. D. (2002) Modern Applied Statistics with S. Fourth edition. Springer) along with least squares regression to estimate the intercept and slope of a line (change in cognitive score=intercept+slope*biomarker). The ‘rlm’ function with ‘psi=psi.biweight’ and method=‘MM’ from the software package R 2.7.1 (R Development Core Team (2008), R: A language and environment for statistical computing. R Foundation for Statistical Computing, Vienna, Austria. ISBN 3-900051-07-0, URL, http://www.R-project.org) was used to compute the estimates for the prediction model.

The prediction of long term (>2 year) change in cognitive score based on a biomarker was determined using a non-linear statistical model. The nonlinear model fit was a three-parameter logistic function: CAMCOG=asymptote/[1+exp([age−xmid]/scale)], in which the xmid parameter is the age at which patients reach 50% of the asymptotic score and the scale parameter is the time taken to fall from three-fourths of the asymptotic score to half the asymptotic score (Martins, et al. (2005) APOE alleles predict the rate of cognitive decline in Alzheimer disease, A nonlinear model, Neurology 2005, 65, 1888-1893). The model was fit using the “nlmixed” procedure in R (R Development Core Team (2008). R: A language and environment for statistical computing. R Foundation for Statistical Computing, Vienna, Austria. ISBN 3-900051-07-0, URL, http://www.R-project.org). The effect of each biomarker was assessed by modeling an interactive effect of the biomarker with the age parameter.

What is claimed:

1. A method for predicting cognitive decline in an Alzheimer's disease (AD) patient comprising:
(a) conducting a biomarker analysis of a fluid sample from an AD patient; and
(b) using the biomarker analysis of step (a) to derive the rate of cognitive decline for the AD patient on a statistically significant slope (SSS) obtained from a standard AD patient panel.

2. The method of claim 1 where the biomarker is selected from the group consisting of Aβ42, tau, and ptau.

3. The method of claim 1 where the predicted cognitive decline is a short-term prognosis.

4. The method of claim 1 where the predicted cognitive decline is a long-term prognosis.

5. A method for evaluating the effectiveness of an Alzheimer's disease (AD) therapeutic comprising:
(a) conducting a cognitive assessment of an AD patient and a biomarker analysis of a fluid sample from the AD patient;
(b) using the biomarker analysis of step (a) to derive the predicted rate of cognitive decline for the AD patient on a statistically significant slope (SSS) obtained from a standard AD patient panel;
(c) administering an AD therapeutic to the AD patient;
(d) conducting a cognitive assessment of the AD patient; and
(e) comparing the result of step (d) with the predicted rate of cognitive decline of step (b).

6. The method of claim 5 where the biomarker is selected from the group consisting of Aβ42, tau, and ptau.

7. The method of claim 1 where the predicted cognitive decline is a short-term prognosis.

8. The method of claim 1 where the predicted cognitive decline is a long-term prognosis.

9. A method for evaluating the relative effectiveness of multiple Alzheimer's (AD) therapeutics comprising:
(a) conducting a cognitive assessment of a group of AD patients and a biomarker analysis of a fluid sample from the patients;
(b) using the biomarker analysis of step (a) to derive the predicted rate of cognitive decline for the AD patient on a statistically significant slope (SSS) obtained from a standard AD patient panel;
(c) dividing said group of AD patients into multiple subgroups having similar predicted rates of cognitive decline;
(d) administering a different AD therapeutic to each subgroup of AD patients;
(e) conducting a cognitive assessment on each subgroup of AD patients;
(f) comparing the results of step (e) with the derived predicted rate of cognitive decline of step (b). for each subgroup.

10. The method of claim 9 where the biomarker is selected from the group consisting of Aβ42, tau, and ptau.

11. The method of claim 9 where the predicted cognitive decline is a short-term prognosis.

12. The method of claim 9 where the predicted cognitive decline is a long-term prognosis.
